(19) United States
(12) Patent Application Publication
(10) Pub. No.: US 2011/0309107 A1
(43) Pub. Date: Dec. 22, 2011

(54) PRESSURIZED RESERVOIR SYSTEM FOR STORING AND DISPENSING LIQUIDS

(75) Inventors: Avraham Shekalim, Nesher (IL); Noam Peleg, Gan-Ner (IL)

(73) Assignee: G-SENSE LTD., Tirat Carmel (IL)

(21) Appl. No.: 13/148,316

(22) PCT Filed: Feb. 25, 2010
(86) PCT No.: PCT/IB10/50833

§ 371 Date: Aug. 8, 2011

Related U.S. Application Data
(60) Provisional application No. 61/155,530, filed on Feb. 26, 2009.

(51) Int. Cl. B67D 7/70

(52) U.S. Cl. 222/135

(57) ABSTRACT

A pressurized reservoir system for storing and dispensing a plurality of liquids in very small quantities in which the liquids are dispensed independently from each other and also together in fixed volumetric proportions.

### FIELD AND BACKGROUND OF THE INVENTION

The present invention generally relates to a pressurized reservoir system for storing and dispensing a plurality of liquids in very small quantities and, particularly, is concerned with two separate liquid dispensing schemes. The first dispensing scheme involves dispensing the liquids independently from each other and the second scheme involves dispensing them together in fixed volumetric ratios.

The difficulty precision dispensing of liquids in miniaturized liquid delivery systems is well-known. Some situations require liquids to be delivered independently while other situations require them to be delivered simultaneously in fixed volumetric proportions. PCT publication WO 2008/056363 discloses a particularly efficient delivery system for delivery of a plurality of liquids simultaneously in fixed volumetric proportion. However, it does not teach a method or system for delivering a plurality of liquids independently. Therefore, there is a need for a liquid delivery system capable of dispensing a plurality of liquids on an independent basis and also simultaneously in fixed volumetric proportion.

### SUMMARY OF THE INVENTION

The present invention is a pressurized reservoir system for storing and dispensing liquids.

According to the teachings of the present invention there is provided, a pressurized reservoir system for storing and dispensing liquids comprising: (a) a housing, (b) a piston arrangement in the housing including at least one piston, the piston arrangement at least partially defining at least two liquid-storage volumes not in fluid communication with each other, each of the liquid-storage volumes having a flow path for dispensing a stored liquid, and (c) a resilient biasing element configured to bias the piston arrangment to pressurize the liquid-storage volumes, wherein the piston arrangement is configured to independently dispense each of the liquids stored in the liquid-storage volumes.

According to a further feature of the present invention, the piston arrangement comprises two pistons in axial alignment, the biasing element being disposed between the two pistons so as to bias both of the two pistons in opposite directions.

According to a further feature of the present invention, the piston arrangement further comprises at least one floating.

According to a further feature of the present invention, the piston arrangement comprises at least one piston having a cavity of parallel walls, wherein the parallel walls slidingly engage an extended body thereby at least partially defining a first of the two liquid-storage volumes.

According to a further feature of the present invention, the at least one piston has an external surface extending from the parallel walls so as to be in sliding engagement with the housing thereby partially defining a second of the two liquid-storage volumes not in fluid communication with each other.

According to a further feature of the present invention, the at least one piston is implemented as a floating piston.

According to a further feature of the present invention, the extended body includes a static projection from the housing.

According to a further feature of the present invention, the static projection includes flow path of the first liquid-storage.

According to a further feature of the present invention, the housing comprises a cylindrical wall.

According to a further feature of the present invention, the biasing element comprises a spring.

According to a further feature of the present invention, the biasing element comprises a compressed gas.

According to a further feature of the present invention, there is also provided a valve-actuator control-system configured for controlling valves regulating flow in the flow paths.

According to a further feature of the present invention, there is also provided an outlet conduit at least partially containing one of the flow paths, the outlet conduit passing through an opening in the primary piston and extending into one of the liquid-storage volumes, the primary piston being in sealed sliding engagement with the outlet conduit.

### BRIEF DESCRIPTION OF THE DRAWINGS

The invention is herein described, by way of example only, with reference to the accompanying drawings, wherein:

FIG. 1 is an isometric, cross-sectional side view of a reservoir system depicting a plurality of reservoirs in a pre-dispensing state.

FIG. 2 is an isometric, cross-sectional side view of the reservoir system after independent dispensing state of one liquid, but prior to linked-dispensing of liquids remaining in the reservoir system.

FIG. 3 is an isometric, cross-sectional side view of an alternative embodiment of the reservoir system including a control system depicting the system before a biasing spring has not been loaded.

FIG. 4 is an isometric, cross-sectional side view of the reservoir system of FIG. 1 pre-dispensing state with the biasing spring loaded and reservoirs filled.

FIGS. 5-7 are schematic, cross-sectional side-views of an alternative embodiment of a reservoir system employing a common biasing spring disposed between two pistons at pre-dispensing, post-independent, intermediate and post-dispensing stages, respectively.

### DESCRIPTION OF THE PREFERRED EMBODIMENTS

An embodiment of the present invention is a miniaturized reservoir system for storing and dispensing very small amounts of liquid in a combination dispensing scheme. The system dispenses at least two liquids independently from each other and, optionally, also dispenses liquids in predefined, fixed volumetric-ratios.

The principles and operation of miniaturized reservoir system according to the present invention may be better understood with reference to the drawings and the accompanying description.

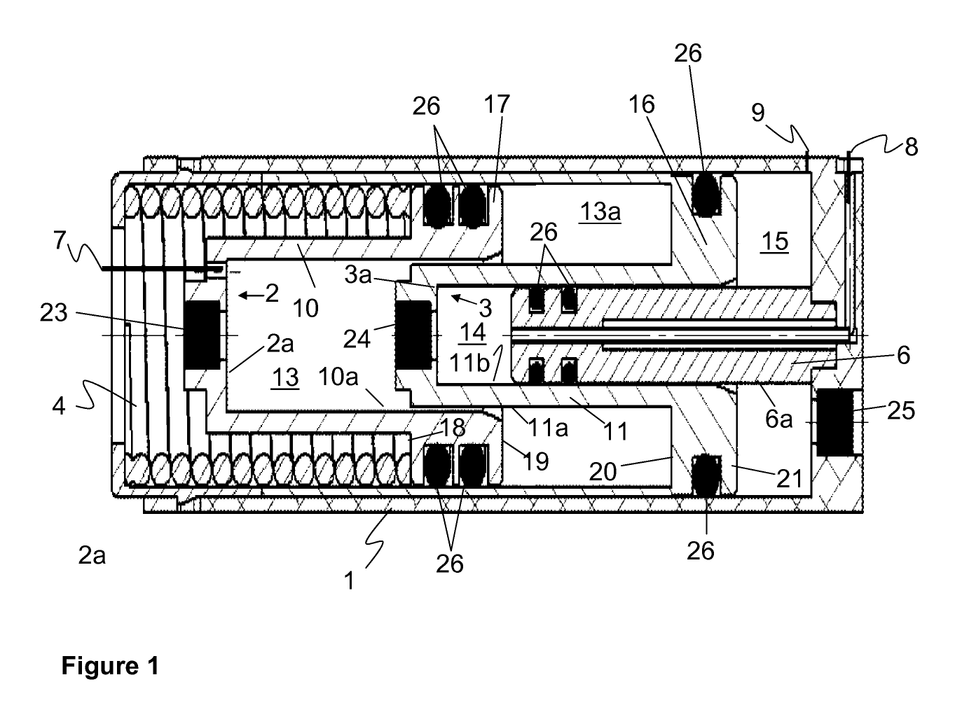

Referring now to the drawings, FIG. 1 depicts a non-limiting, exemplary embodiment of a reservoir system in an initial pre-dispensing state in which each reservoir is fully supplied with an appropriate liquid. It should be noted that for the sake of convenience the present document discusses the reservoir system in the context of a combined insulin infusion and glucose monitor based on a microdialysis probe arrangement; however, it should be appreciated that any system requiring storage and precise delivery of a plurality of liquids is included within the scope of the present invention. The reservoir system includes a system housing 1, a primary piston 2 in axial alignment with a secondary piston 3 forming an overall piston arrangement, a spring 4 for biasing the piston arrangement to advance in housing 1, a static projection 6, a primary flow path 7, a secondary flow path 8 which extends in part along static projection 6, and a tertiary flow path 9. Primary piston 2 includes a parallel piston wall 10, typically of cylindrical form and terminating in a closure 2a, that forms a primary cavity 13. Similarly, secondary piston 3 includes a parallel piston wall 11, typically of cylindrical form and terminating in a closure 3a, forming a secondary cavity 14. The diameter of a cylindrical external surface 11a of wall 11 is less than the diameter of a cylindrical internal surface 10a of wall 10 so that primary and secondary pistons 2 and 3 can nest. Optionally, the two cylindrical walls may be configured to engage in sliding engagement, but preferably with flow channels formed in one or both surfaces to interconnect volumes 13 and 13a. Alternatively, there may be a clearance there between. Static projection 6 is parallel walled, and preferably cylindrical, with an outer surface 6a configured to engage and seal against the inner surface 11b of wall 11, thereby isolating storage volumes 14 and 15 from each other. Primary cavity 13 and secondary cavity 14 each serve as liquid storage volumes not in fluid communication with each other because they are divided by floating, secondary piston 3 thereby enabling liquids to be dispensed from primary storage volume 13 while liquids in secondary storage volume 14 remain in storage as will be further discussed. The above-described piston arrangement is in axial alignment with spring 4 disposed at one end of housing 1.

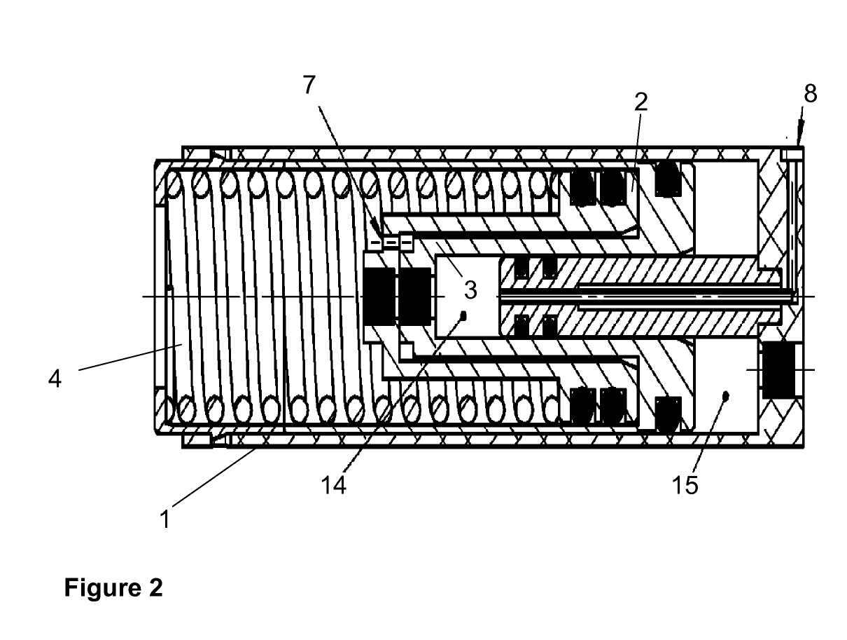

At this point it is helpful to define some terms of reference used throughout the present document.
“Fluid Communication” refers to a flow arrangement between a plurality of liquid storage volumes in which liquid flows from one storage volume to another.
“Parallel Walls” refers to any wall arrangement in which the distance between opposing walls or opposing sections of a single wall is constant.
“Floating Piston” refers to a piston not acted upon by a mechanical linkage but rather is acted upon by liquids in which the piston is disposed.
“Distal” refers to a side most distant from a biasing element.
“Flange” refers to any surface extending outwards from the piston wall towards the housing.
   Primary flange 17 is disposed at a distal end of the primary piston wall 10 and primary flange 17 extends radially to housing 1 where it slidingly engages it. Similarly, secondary flange 16 is disposed at the distal end of piston wall 11 and extends radially to housing 1 where it also slidingly engages it. Seals 26, preferably implemented as “o-rings”, are disposed in between flange surfaces in sliding contact with housing 1 and walls 6a of static projection 6 to ensure a leak-free sliding engagement. Distal surface 19 of primary flange 17 and non-distal surface 20 of secondary flange 16 partially define an additional liquid storage volume 13a that in the present non-limiting embodiment is in fluid communication with primary storage volume 13. Distal surface 21 of secondary flange 16 partially defines tertiary liquid storage volume 15. In a non-limiting exemplary embodiment, primary flow path 7 (shown here schematically) is in fluid connection with primary storage volume 13 and passes through the space circumscribed by spring 4 as shown in FIGS. 1 and 2. Secondary flow path 8 is fluid communication with secondary liquid storage volume 14 and passes through static projection 6 as mentioned above. Tertiary flow path 9 is in fluid communication with tertiary liquid storage volume 15. As mentioned above, spring 4 is disposed at one end of housing 1 and applies a bias to the piston arrangement. In this particular, non-limiting embodiment, spring 4 resiliently bears directly on non-distal surface 19 of primary flange 2 thereby pressurizing liquids in volume 13, and indirectly, by the liquid pressure acting on surfaces of the floating secondary piston 3, also pressurizes liquids in secondary and tertiary volumes 14 and 15, respectively. Each flow path typically includes a valve and valve actuator (not shown) controlled by a control system in accordance with system parameters; closed valves maintain the liquids in liquid-storage volumes and open valves dispense the liquids as is known in the art. It should be noted that, in a non-limiting, exemplary embodiment, the housing is implemented as a cylindrical wall. However, non-cylindrical embodiments are included within the scope of the present invention.

FIG. 2 depicts the reservoir system after dispensing of the liquid stored in the combined storage volumes 13 and 13a, but where the liquids in storage volumes 14 and 15 have not yet been dispensed. It will be noted that this state has been chosen for clarity of presentation, but there is no limitation as to the sequence and relative rates of dispensing of the liquid in storage volume 13, 13a versus that in storage volume 14. Independent dispensing commences when a valve in flow path 7 is opened. Primary piston 2, biased by spring 4, advances in housing 1 and expels liquids stored liquid-storage volume 13 as without affecting liquids stored in other storage volumes 14 or 15. This independent type of liquid dispensing advantageously provides selective control of liquid delivery. In combined drug delivery and diagnostic testing applications, such as in a system for delivering insulin and performing microdialysis blood glucose analysis, such delivery schemes have special significance; insulin may be stored in liquid storage volume 13 and dispensed independently while dialysate (such as saline solution) and a reagent are held in stored and dispensed from volumes 14 and 15, preferably in a linked dispensing mode to be described.

Linked-dispensing commences when valves disposed in flow paths 8 and 9 are opened. Secondary piston 3 and its distal flange surface 21 advance in unison in housing 1 thereby expelling liquids simultaneously from storage volumes 14 and 15. Storage volumes 14 and 15 are the same length to ensure that secondary piston 3 and its flange 16 fill each storage volume entirely when they reach a fully displaced position at the end of their range of movement. Given the constant length of volumes 14 and 15, the cross-sectional area of each of each storage volume defines the amount of liquid expelled as piston 3 advances. Accordingly, the ratio of the cross-sectional surfaces of storage volumes 14 and 15 defines the volumetric ratio at which the liquids are dispensed. In the above mentioned combined insulin administration and microdialysis unit, this feature again has special significance because saline solution and reagent, stored in either of storage volumes 14 and 15, are dispensed in the required, fixed volumetric ratio. In some cases, control of both flow rates may be achieved by controlling a valve in only one of the outlet flow paths while the other remains continuously open. Since liquid is only released from storage volumes 14 and 15 in fixed volumetric ratio, neither liquid flows unless both flow paths are open. It should be appreciated that embodiments in which linked dispensing precedes independent dispensing or both dispensing schemes are performed simultaneously are included within the scope of the present invention. Replacement liquid is injected into primary liquid storage volumes 13 and 13a through septum 23 and similarly, additional replacement liquid is injected into liquid storage volume 15 through septum 25. However, given that storage volumes 14 is non-contiguous with housing 1, replacement liquid is introduced by way of an extended needle, or similar instrument, capable of spanning the entire length of storage-volume 13 and piecing septum 24. Additional issues regarding the refilling of the storage reservoirs will be discussed later in the document.

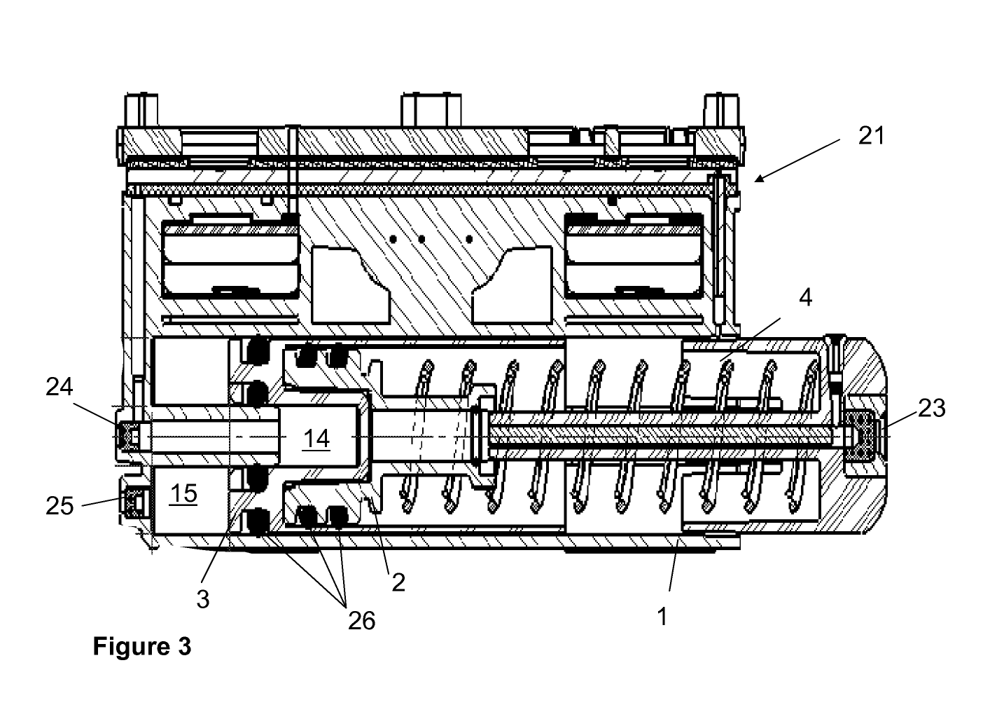

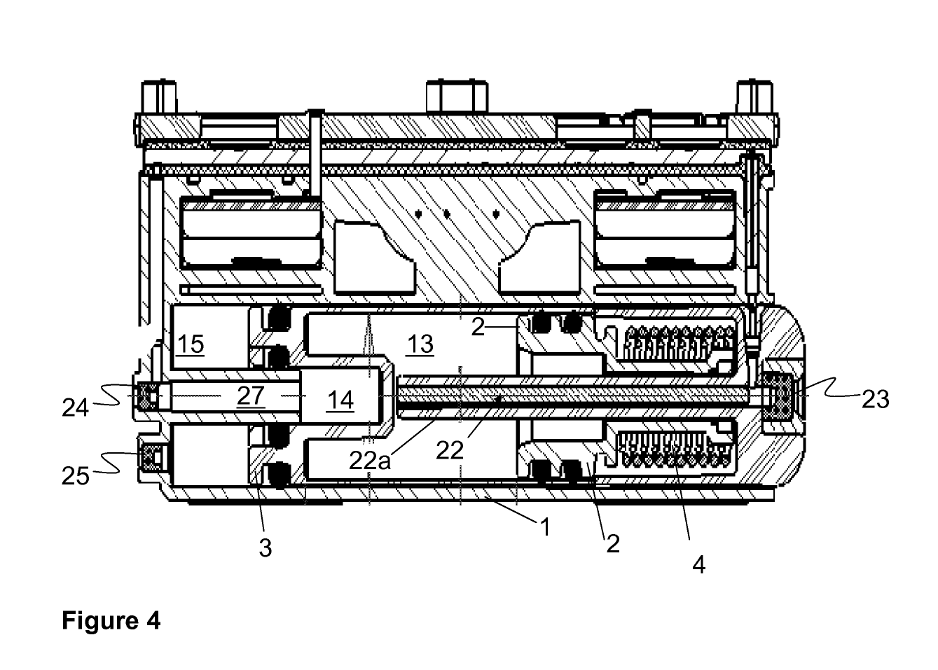

FIGS. 3 and 4 depict a non-limiting, alternative embodiment including an associated valve control arrangement 21. Generally speaking, the reservoir system of this embodiment is similar to that of FIGS. 1 and 2, and equivalent elements are designated similarly. Any suitable control system 21 may be used. PCT publication WO 2004/105827 discloses a non-limiting example of an appropriate control system; FIGS. 6-14 of the aforementioned PCT publication and their associated description are hereby incorporated by reference herein. FIG. 3 further depicts biasing element in a non-loaded state. It should be appreciated that biasing element 4 may be implemented as any suitable biasing element. Exemplary embodiments illustrated here employ helical, compression springs; however, helical tension springs or non-helical springs constructed from metallic or non-metallic materials are included within the scope of the present invention. Furthermore, it should be noted that air springs or compressed gas equivalents are also included within the scope of the present invention.

FIG. 4 depicts the above-mentioned alternative embodiment in a spring loaded, filled state prior to dispensing. A flow path 22 is disposed in a flow conduit 22a projecting through the space circumscribed by spring 4, an opening in primary piston 2, and through the majority of primary liquid storage volume 13. Liquids in primary storage volume 13 are expelled through a flow path inlet disposed at the distal end of storage volume 13. Primary piston 2 is in leak free, sliding engagement with flow path 22.

The storage reservoirs of each embodiment are refilled with the appropriate liquids by way of their flow paths and/or by suitably positioned septum seals. For example, liquid is injected though septum 23 into flow path 22 and into primary liquid storage volume 13. Similarly, the appropriate liquids are injected through septum 25 directly into liquid storage volume 15 and through septum 24 into flow path 27 leading into liquid storage volumes 14. It should be noted that, when refilling storage volumes involved in linked dispensing, both liquids most be replenished simultaneously to ensure that each volume is entirely filled. A void remaining in either of the storage volumes will distort the fixed volumetric ratio at which the liquids are to be delivered. Therefore, each liquid must be injected into the relevant storage volumes simultaneously at flow rates corresponding to the volumetric flow ratio defined by the structure, as discussed above. In practical terms, one replacement liquid is preferably injected into a first storage volume while the remaining flow path is in sealed, air tight connection with the second replacement liquid. The injected replacement liquid fills the storage volume, pushing the piston and its associated flange in unison thereby creating a partial vacuum that draws in the second liquid as the piston retracts.

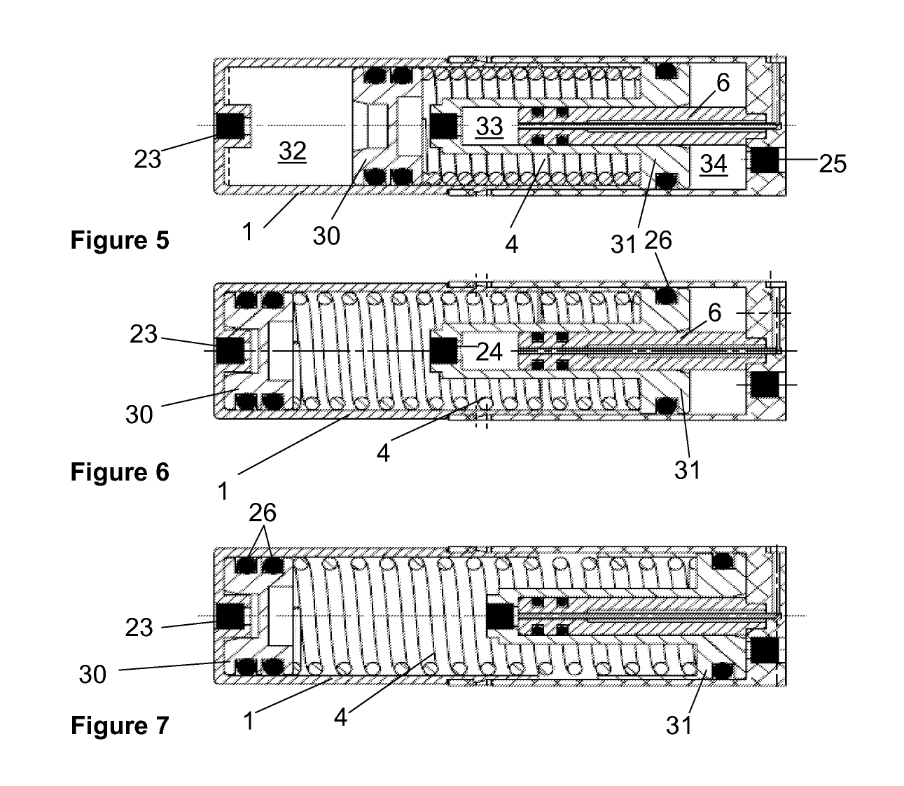

FIGS. 5-7 depict a third, structurally analogous, embodiment having resilient biasing spring 4 disposed in between axially aligned rearward piston 30 and forward piston 31, biasing each piston towards its corresponding end of the housing. Rearward piston 30 partially defines liquid storage volume 32 that is not in fluid communication with liquid storage volumes 33 and 34. As rearward piston 30 advances in a rearward direction (to the left as shown), liquid held in storage volume 32 is dispensed independently of the liquids held in storage volumes 32 and 33. FIG. 6 shows the state of the device after the liquid from volume 32 is completely dispensed. Forward piston 31 partially defines volumes 32 and 33 also not in fluid communication with each other, so that as forward piston 31 moves in a forward direction (to the right as shown), liquids held in both storage volumes 32 and 33 are dispensed simultaneously in a fixed, volumetric ratio as described above. FIG. 7 shows the empty device after all liquids have been dispensed.

It will be appreciated that the present invention is of particular advantage in any application where two or more liquids must be stored in a compact volume and be supplied under pressure. As mentioned above, the present reservoir system has particular application in regards to a combined Continuous Glucose Measurement (CGM) and Continuous Subcutaneous Insulin Infusion (CSII) system. However, it should be noted that the system has application in the administration of insulin with glucagon or GLP (Glucagon-Like Peptide), or insulin with other drugs and enzymes, and also for independent delivery of liquids and fluid mixing.

The reservoir system and its various components may be constructed from any suitable materials including, but not limited to, polymeric materials and metallic materials as is known in the art.

It will be appreciated that the above descriptions are intended only to serve as examples, and that many other embodiments are possible within the scope of the present invention as defined in the appended claims.

What is claimed is:

1. A pressurized reservoir system for storing and dispensing liquids comprising;
(a) a housing;
(b) a piston arrangement in said housing including at least one piston, said
piston arrangement at least partially defining at least two liquid-storage volumes not in fluid communication with each other, each of said liquid-storage volumes having a flow path for dispensing a stored liquid; and
(c) a resilient biasing element configured to bias said piston arrangment to pressurize said liquid-storage volumes,
wherein said piston arrangement is configured to independently dispense each of the liquids stored in said liquid-storage volumes.

2. The pressurized reservoir system of claim 1, wherein said piston arrangement comprises two pistons in axial alignment, said biasing element being disposed between said two pistons so as to bias both of said two pistons in opposite directions.

3. The pressurized reservoir system of claim 1, wherein said piston arrangement further comprises at least one floating.

4. The pressurized reservoir system of claim 1, wherein said piston arrangement comprises at least one piston having a cavity of parallel walls, wherein said parallel walls slidingly engage an extended body thereby at least partially defining a first of said two liquid-storage volumes.

5. The pressurized reservoir system of claim 4, wherein said at least one piston has an external surface extending from said parallel walls so as to be in sliding engagement with said housing thereby partially defining a second of said two liquid-storage volumes not in fluid communication with each other.

6. The pressurized reservoir system of claim 4, wherein said at least one piston is implemented as a floating piston.

7. The pressurized reservoir system of claim 5, wherein said extended body includes a static projection from said housing.

8. The pressurized reservoir system of claim 5, wherein said, wherein said static projection includes flow path of said first liquid-storage.

9. The pressurized reservoir system of claim 1, wherein said housing comprises a cylindrical wall.

10. The pressurized reservoir system of claim 1, wherein said biasing element comprises a spring.

11. The pressurized reservoir system of claim 1, wherein said biasing element comprises a compressed gas.

12. The pressurized reservoir system of claim 1, further comprising a valve-actuator control-system configured for controlling valves regulating flow in said flow paths.

13. The pressurized reservoir system of claim 1, further comprising an outlet conduit at least partially containing one of said flow paths, said outlet conduit passing through an opening in said primary piston and extending into one of said liquid-storage volumes, said primary piston being in sealed sliding engagement with said outlet conduit.
